(19) United States
(12) Patent Application Publication
(10) Pub. No.: US 2018/0058945 A1
(43) Pub. Date: Mar. 1, 2018

(54) MICROWAVE RADIOMETER

(71) Applicant: LIMITED LIABILITY COMPANY "RTM DIAGNOSTICS", Moscow (RU)

(72) Inventor: Sergey Georgievich VESNIN, Moscow (RU)

(21) Appl. No.: 15/801,419

(22) Filed: Nov. 2, 2017

Related U.S. Application Data
(63) Continuation of application No. PCT/RU2015/000953, filed on Dec. 29, 2015.

(30) Foreign Application Priority Data
Dec. 22, 2015 (RU) 2015154996

(51) Int. Cl. G01K 11/00; A61B 5/01; A61B 5/05; G01K 13/00

(52) U.S. Cl. CPC G01K 11/006; G01K 13/002; A61B 5/0507; A61B 5/015

(57) ABSTRACT

Radiometer for non-invasive measurement of internal tissue temperature of biological objects. The radiometer comprises, connected in series, antenna, SPDT switch, circulator, receiver including amplifier with bandpass filters, amplitude detector, narrowband low-frequency amplifier and synchronous detector, integrator, direct current power amplifier, reference voltage generator connected to the SPDT switch and synchronous detector. A Peltier element is connected to the receiver output. First and second microwave loads are installed on the Peltier element and have thermal contact with it. There is at least one temperature sensor for measuring the temperature of microwave loads. The first microwave load is adapted for connection to the SPDT switch. The SPDT switch is adapted to connect either, to a first arm of the circulator, the antenna, or the first microwave load. A second arm of the circulator is connected to the receiver, and a third arm of the circulator is connected to the second microwave load.

### CROSS-REFERENCE TO RELATED APPLICATIONS

The present application is a continuation of PCT/RU2015/000953 filed on Dec. 29, 2015. This application also claims the benefit of Russian Patent Application RU 2015154996 filed on Dec. 22, 2015. The contents of the aforementioned applications are incorporated by reference herein.

### FIELD

Non-limiting embodiments of the present technology relate to the field of medicine and medical equipment, namely, to radiometric techniques based on non-invasive detection of thermal abnormalities of the internal tissue of biological objects by measuring the intensity of their own electromagnetic radiation in general and in particular to a microwave radiometer for non-invasive measurement of the temperature of internal tissue of a biological object.

The present technology can be used in medical equipment for non-invasive measurement of the internal tissue temperature, temperature monitoring, identification of temperature variations and thermal abnormalities of internal tissues of a biological object in diagnostic complexes for early diagnosis of oncological diseases.

### BACKGROUND

RU Pat. No. 2082118 “Medical Radiometer” to A. V. Vaisblat describes a null balancing radiometer. The block diagram corresponding to that radiometer is shown in FIG. 1. The radiometer includes an microwave antenna (1), which contacts with a biological object to detect the signal of thermal noise emitted by a biological object or a portion of biological object to determine the temperature thereof. From the antenna output the noise signal, whose power is proportional to the brightness of the biological object's temperature, comes to a modulator (2). For loss-less antennae the temperature of noises at the antenna output coincides with the biological object's temperature (the brightness temperature of the biological object). If the antenna has thermal losses, then thermal noises of the antenna itself are added to the noise temperature at the antenna output.

RU Pat. No. 2082118 describes a modulator which is controlled by reference voltage generator (3) and it performs the function of a SPST switch (single-ode, single-throw switch), that is when the modulator is on, the noise signal from an antenna output comes to circulator (4) and then to an input of receiver (5), and when the modulator is off, the noise signal which power is proportional to noise temperature Tᵣ of heated resistor (6) comes to the input of the receiver.

A receiver (5), consisting of a low-noise amplifier including bandpass filters (7) (BP), an amplitude detector (8), a low frequency (LF) amplifier and a selective amplifier (9), a synchronous detector (10), an integrator (11), and a direct current (DC) amplifier (12), forms voltage proportional to the difference of noise temperature Ta of the antenna output and noise temperature of the heated resistor. This voltage comes on a heated resistor (6), which leads to thermodynamic temperature change and, consequently, the noise temperature of the heated resistor changes because the noise temperature of heated resistor Tr coincides with its thermodynamic temperature in the absence of reflections. Due to negative feedback, voltage at the synchronous detector output ΔU tends toward zero while noise temperature Tᵣ of the heated resistor tends toward noise temperature Tₐ from the antenna output.

That means that the task of measuring microwave power coming from the antenna output is replaced with the task of measuring temperature of the heated resistor and maintaining voltage at the synchronous detector output close to zero. Temperature of the heated resistor is measured using a temperature sensor (13), installed on the heated resistor. To reduce the fluctuation error, the voltage coming from the temperature sensor output is averaged in an integrator (14) during time T, is amplified and then comes onto the indicator or for transmission to a computer.

The error of measurement of brightness temperature in microwave radiometers depends on several factors. Firstly, due to dissipation losses of the microwave radiometer frontend, noise power from the antenna output and thermal noise of radiometer frontend (circulator and switch) enter to the receiver input, and it is defined as:

ΔT=∝_{dis}*(T_{amb}−Tₐ), where
ΔT is the error of measurement of the brightness temperature due to dissipation losses of the microwave radiometer frontend,
T_{amb} is the noise temperature of the radiometer frontend (circulator and switch),
Tₐ is the noise temperature of the antenna output,
∝_{dis} is equivalent dissipation losses of the radiometer frontend,
therefore, the temperature of the radiometer frontend influences on the measurement results. At a first approximation for modulated radiometers shown in FIG. 1, the following equation is true

$${∝}_{dis} = \frac{{∝}_{sw} - {∝}_{cir}}{1 - {∝}_{sw}} ,$$

where
∝_{cir} is the circulator's dissipation losses,
∝_{sw} is the modulator dissipation losses.

To reduce the error related to temperature change of the radiometer's frontend, dissipation losses of the circulator and modulator must be balanced. In practice, this is rather difficult to provide since the dissipation losses of components of microwave radiometer have a certain dispersion, and adjustment of the dissipation losses is rather complicated.

It follows from the abovementioned formulae that an increase of circulator attenuation by 0.3 dB entails an error of measurement of the biological object's temperature of 1° C. when the temperature of the radiometer's frontend changes by 20° C.

Besides, medical microwave radiometers have an error of measurement of the brightness temperature related to the fact that the incoming impedance of a biological object might vary within a rather wide range while the input resistance of the antenna is fixed. As a result, the antenna lacks ideal matching and has reflection coefficient R, so a part of the thermal noise signal from a biological object is reflected from the antenna and does not enter the receiver. In particular, if the antenna is matched for a tissue having dielectric permeability E equal to 10, then during measurement of the temperature of a muscular tissue having E equal to 40, the reflection coefficient will be equal to 0.33 and 10% of the power signal will be reflected from the antenna, which, correspondingly, might result in an error of brightness temperature measurement equal to 30 K.

For compensation of this error related to reflection, U.S. Pat. No. 4,235,107 A published 25 Nov. 1980 to Ludeke describes schemes compensating power losses related to the end reflection coefficient by using an additional source of noise.

Similarly, in RU Pat. No. 2082118, as shown on the block diagram given in FIG. 1, compensation of reflections is achieved due to that noise from the heated resistor (6) passing through the circulator (4) and entering the antenna. If there are reflections from the antenna, a part of the heated resistor's power noise is reflected from the antenna and goes back into the receiver, thus compensating the power reflected from the antenna. Obviously, if the noise temperature of a biological object is equal to the noise temperature entering the antenna from the heated resistor, then the reflected power will be fully compensated. But as the noise temperature of a biological object varies within a certain range it is rather difficult to fulfil this condition and the error of measurement due to signal reflection is usually quite significant. Even if the equality of noise temperatures from the antenna output and from the resistor is achieved, that is Ta=Tr, due to thermal losses in the circulator and modulator, the power of noise entering the antenna on the side of the heated resistor is not equal to the power of noise coming from a biological object, so full compensation does not occur.

On the block diagram shown in FIG. 1, taken from RU Pat. No. 2082118, the circulator (4) is designed not only for compensation of reflections from the antenna, but also for increase isolation between the receiver and antenna. Noise coming from the receiver input is absorbed in the circulator's balance resistor and does not enter the antenna. Isolation of real circulators is usually not high enough to suppress fully the noise signal coming from the receiver input. As a result, receiver's noise that has passed through the circulator is reflected from the OFF switch and enters again the receiver input.

Referring to the radiometer described by Vaisblat A. V. in the paper “Medical Radiometer RTM-01-RES”, Biomedical Technologies and Radio Electronics, No. 8, 2001, P. 11-23, the block diagram of FIG. 2 contains an antenna (1) contacting a biological object, a modulator (2), which performs the function of a SPST switch (2), a circulator (4), and a receiver (5) including a low-noise amplifier with bandpass filters (7), an amplitude detector (8), a low-frequency amplifier and a selective amplifier (9), a synchronous detector (10), an integrator (11), and a direct current amplifier (12), also a reference voltage generator (3). In the prototype radiometer, to increase isolation between the receiver (5) and the modulator, after the circulator (4), one more nonreciprocal element—a isolator (15)—is installed (see FIG. 2). However, nonreciprocal elements have considerable dimensions and high price compared to other elements of a radiometer; therefore, to reduce radiometer dimensions and price, it is necessary to decrease the number of nonreciprocal elements. Besides, the radiometer uses a Peltier element (16) to change the heated resistor temperature.

However, the design shown in FIG. 2 does not provide sufficient accuracy of measurement either because due to dissipation losses in the circulator and modulator, the power of noise entering the antenna on the side of heated resistor is not equal to the power of noise coming from a biological object so full compensation of reflections at the interface of media does not occur and the accuracy of measurement is still insufficient.

### SUMMARY

An object of the present technology is to create a null balancing radiometer for non-invasive detection of temperature abnormalities of internal tissues, which has a small error of brightness temperature measurement and a minimal number of nonreciprocal elements.

The solution of the said problem ensures reduction of the error of measurement of the internal temperature of a biological object and improved accuracy of the device in finding malignant tumors, also decreased dimensions of the instrument, better convenience of using, and lower cost of its manufacture.

From one aspect, there is provided a radiometer for non-invasive detection of temperature abnormalities of internal tissues. The radiometer contains connected in series: an antenna for contact with a biological object; a SPDT switch (a single-pole double-throw switch); a circulator optionally installed after the SPDT switch; a receiver including an amplifier with bandpass filters, an amplitude detector, a narrowband low-frequency amplifier, a synchronous detector, an integrator, a direct current power amplifier, and a reference voltage generator which is connected to the SPDT switch and to the synchronous detector; a Peltier element connected to an output of the receiver, first and second microwave loads mounted on the Peltier element and thermally contacting it, at least one temperature sensor for measuring the temperature of the first and second microwave loads, wherein the first microwave load is adapted for connection to the SPDT switch, the SPDT switch is adapted to connect either the antenna or the first microwave load to a first arm of the circulator, a second arm of the circulator being connected to the receiver, and a third arm of the circulator being connected to the second microwave load.

In one embodiment, the radiometer may additionally include an attenuator mounted between the output of the first microwave load and the SPDT switch.

The temperature sensor made with the faculty of measuring microwave load temperature may be installed on the Peltier element and/or on a microwave load.

The temperature sensor may be an infrared temperature sensor, which is arranged to measure remote temperature, and/or a temperature sensor installed on microwave loads and/or the Peltier element and having good thermal contact with them.

The radiometer may also include an additional integrator connected to the outlet of at least one temperature sensor.

All elements of the radiometer, including the circulator, Peltier element and SPDT switch, are mounted on a heat-conductive base and have a thermal contact with it, so, the temperature of all elements of the radiometer frontend (the SPDT switch, circulator, attenuator) is close to the temperature of the base they are mounted on.

So, the first side of the Peltier element is installed on the base and has a good thermal contact with it, while two microwave loads are installed on the Peltier element side that is opposite to the base, and have a good thermal contact with it.

All components of the microwave radiometer have a common microwave signal earthing.

### BRIEF DESCRIPTION OF THE DRAWINGS

FIG. 1 is a block diagram of the known from the prior art, null balancing radiometer according to RU Pat. No. 2082118.

FIG. 2 is a block diagram of the known from the prior art, null balancing radiometer according to the closest analogue (prototype) of the present technology, which has two nonreciprocal elements and in which the resistor is accommodated on a Peltier element.

FIG. 3 is a block diagram of an embodiment of a radiometer, according to the present technology, which has a SPDT switch and two microwave loads installed on a Peltier element and connected, correspondingly, to the circulator and SPDT switch.

FIG. 4 is a block diagram of another embodiment of the radiometer, according to the present technology, with a SPDT switch and two microwave loads installed on a Peltier element, wherein an attenuator is mounted between the first microwave load and the SPDT switch.

### DETAILED DESCRIPTION OF THE NON-LIMITING EMBODIMENTS

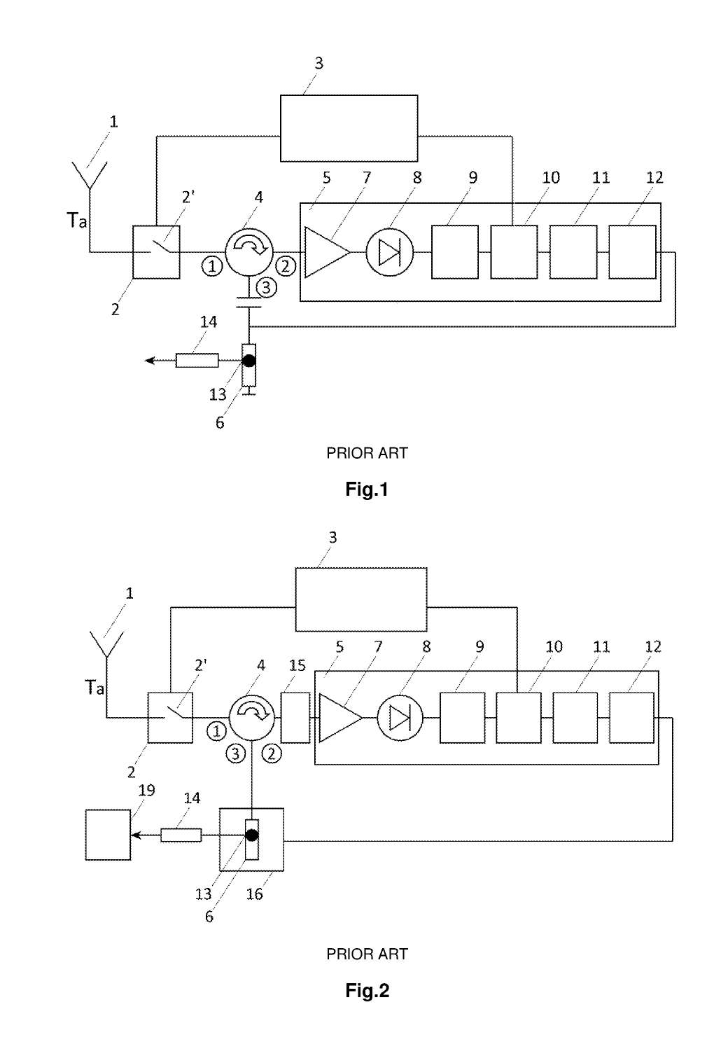

FIG. 1 shows a block diagram of a null balancing radiometer known from the prior art, (RU Pat. No. 2082118), in which the block diagram has been adapted and is presented similarly to the present technology. This known null balancing radiometer consists of an antenna (1) for contact with a biological object and receive a noise signal coming from the biological object. From the antenna output, the microwave noise signal enters the input of an electronic modulator (2). In the modulator (2) of the radiometer according to RU Pat. No. 2082118, a SPST switch (2′) is used, which closes and opens the connection of a circulator with the antenna.

The modulator is controlled by a reference voltage generator with a clock frequency of 1 kHz. When the SPST switch (2′) of the modulator is ON, the noise signal from the antenna output enters a circulator (4) and then enters the input of a receiver (5). When the SPST switch (2′) of the modulator is off, the noise signal from a heated resistor accommodated on the third arm of the circulator (4) is reflected from the OFF switch of the modulator and enters the input of the circulator and then the input of the receiver (5).

The receiver contains a low-noise amplifier with bandpass filters (7), an amplitude detector (8), a narrowband low-frequency amplifier (9), a synchronous detector (10), an integrator (11), a direct current amplifier (12).

At the receiver output, voltage is formed that is proportional to the difference of the heated resistor noise temperature Tr and the temperature Ta of noise coming from the antenna output

ΔU=k(Tₐ−Tᵣ), where
k is the gain of the radiometer receiver.
Ta is the noise temperature from the antenna output,
Tr is the noise temperature of the resistor.

This signal is amplified and comes onto the heated resistor (6), resulting in a change of its thermodynamic temperature and, consequently, the resistor noise temperature Tr. Cooling of the heated resistor was achieved by way of natural air cooling.

Due to negative feedback, voltage at the synchronous detector outlet tends toward zero, and the noise temperature of the heated resistor Tᵣ tends toward the noise temperature Ta coming from the antenna output.

As at the synchronous detector output, voltage is close to zero, the noise temperature coming from the antenna output is equal to the noise temperature of the heated resistor.

In the absence of reflections, the noise temperature Tr of the heated resistor coincides with the thermodynamic temperature measured with the help of a temperature sensor mounted on the heated resistor. To reduce the fluctuation error, voltage coming from the temperature sensor output is averaged in the integrator (14) and amplified.

FIG. 2 shows, in a presentation form similar to the present technology, a block diagram of a commercial radiometer—prototype described by Vaisblat A. V. in paper “Medial Radiometer RTM-01-RES”, Biomedical Technologies and Radio Electronics, No. 8, 2001, P. 11-23. That prototype radiometer consists of an antenna (1), a modulator (2), a circulator (3), an isolator (15), a receiver (5), a Peltier element (16), a microwave load (6), mounted on a Peltier element (16), a temperature sensor (13) measuring the temperature of microwave load (6), an integrator (14), a reference voltage generator (3) controlling the modulator (2). Wherein, similarly to the radiometer of RU Pat. No. 2082118, the Vaisblat prototype modulator (2) includes a SPST switch (2′), which can only close and open the connection between the circulator and antenna.

The receiver in the prototype consists of a low-noise amplifier with bandpass filters (7), an amplitude detector (8), a narrowband low-frequency amplifier (9), and a synchronous detector (10), and integrator (11), direct current amplifier (12).

In the prototype radiometer shown in FIG. 2, in contrast to the block diagram shown in FIG. 1, the load temperature is controlled with the help of Peltier element (16). This allows implementing both heating and cooling of the load. To increase isolation between the receiver and antenna, in the prototype radiometer a second nonreciprocal element—isolator (15)—is mounted. This allows increasing isolation between the SPST switch (2′) and receiver to 34 dB in the frequency spectrum of 500 MHz and reduce the level of noise coming on to the modulator (2) from the receiver (5), but enlarges outer dimensions of the device.

Thus, the prototype radiometer does not provide the required accuracy of measurement because compensation of reflections from the antenna is still insufficient, besides, the radiometer has large outer dimensions due to use of nonreciprocal elements, for example, the isolator (15), which makes it inconvenient in use.

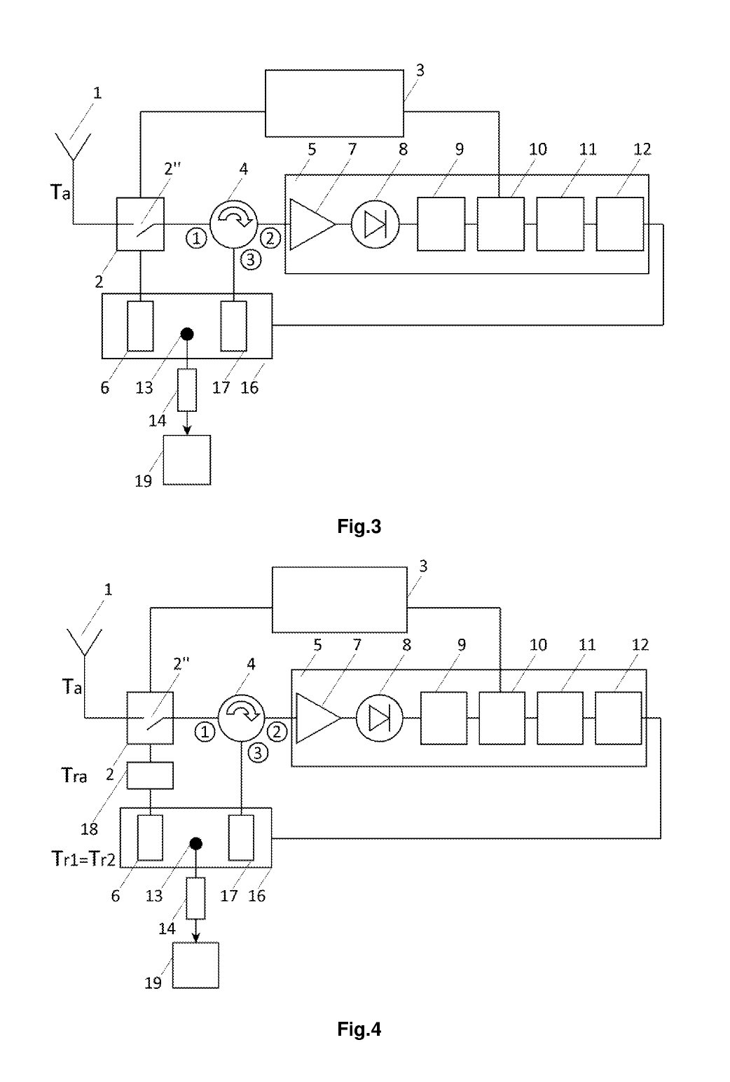

The design of the presently claimed radiometer is explained in detail with a reference to FIGS. 3 and 4. FIG. 3 shows the first embodiment of the radiometer according to the present technology, which additionally has a second microwave load (a second resistor) installed on the Peltier element, and the modulator has a SPDT switch (2″) rather than a SPST switch, which connects to the first arm of the circulator either the antenna (1) or the first load (6) (the first resistor). The SPDT switch (2″) is controlled by a reference voltage generator, for example, having 1 kHz frequency. The noise signal from the output of SPDT switch (2″) passes through a circulator (4) and enters a receiver (5).

The receiver (5) contains a low-noise amplifier with bandpass filters (7), an amplitude detector (8), a narrowband low-frequency amplifier (9), a synchronous detector (10), an integrator (11), a direct current amplifier (12).

During operation of the present radiometer, voltage ΔU is formed at the receiver output, which is proportional to the difference of the noise temperature coming from the antenna and temperature Tr₁ of the first heated resistor:

ΔU=k(Tₐ−Tᵣ₁), where
k is the gain of the radiometer receiver,
Ta is the noise temperature from the antenna output,
Tr₁ is the noise temperature of the first microwave load (the first resistor).

This voltage is amplified and comes on the Peltier element (16). In contrast to the block diagrams of the prior art devices shown in FIG. 1 and FIG. 2, in the present technology, two microwave loads (6) and (17) are used, that is two resistors installed on the Peltier element and having good thermal contact with the Peltier element. The first microwave load (6) is connected to the input of SPDT switch (2″) of the modulator (2). The second microwave load (17) is connected to the third arm of the circulator (4).

The temperature of microwave loads is measured with the help of a temperature sensor (13), which may be mounted on the Peltier element or at least on one of the loads and has a good thermal contact with them, then the measurement signal from the temperature sensor is integrated in an additional integrator (14) connected with the temperature sensor (13), is amplified and comes onto an indicator or into a computer (19), performing the functions of a data processing unit and a control unit.

In contrast with the prior art block diagrams of FIG. 1 and FIG. 2, in the present technology, instead of the SPST switch (2′), which is contained in the modulator and either connects the antenna output with the circulator or breaks the connection, a SPDT switch (2″) is used, which connects either the antenna or the first microwave load to the first arm of the circulator. In this instance, at the input of the receiver (5), comparison of signals coming from the antenna (1) and from the first microwave load (6) takes place.

In some prior art solutions, attempts have also been made to use SPDT switch in radiometer designs, for example, in the design of the radiometer according to RU Pat. 2485462 published 20 Jun. 2013, between a modulator and a circulator, a directional coupler is installed, to which a two-pole switch having three inputs and two outputs is installed. In this design, three matched loads are used, wherein the first matched load is connected to a circulator, and the second and third matched loads may be commutated to the SPDT switch, and the SPDT switch in RU patent 2485462 is made with the faculty of either connecting the first output of the SPDT switch to a noise generator and the second output to the second matched load, or connecting the first output of the SPDT switch of the third matched load and the second output to the noise generator. Whereas, the modulator, directional coupler, circulator, SPDT switch, noise generator and source of current for it, as well as the first, second and third matched loads are mounted on a thermostat plate and have an equal temperature, but there is not Peltier element in this design.

Thus, the radiometer schematic according to the present technology has a simpler design, contains only two matched microwave loads, which have other connections to other elements of the design. Besides, in the presently claimed radiometer, both loads are mounted on a Peltier element, which can both heat loads and cool them, hence, loads have an equal regulated temperature that is different from the temperature of other elements of the schematic. This provides a higher accuracy of brightness temperature measurement at a minimal number of nonreciprocal elements, which reduces the outer dimensions of the design and improves convenience of its use during measurement of the internal temperature in many points of a biological object.

During radiometer operation, due to negative feedback, voltage at the synchronous detector outlet tends toward zero and noise temperature Tᵣ₁ of the first load (6) comes close to the temperature of noise Tₐ coming from the antenna output.

Due to an imperfect isolation of the circulator, a part of the receiver noise passes through the circulator (4) and enters the SPST switch (2″). In the prior art prototype radiometer, which block diagram is shown in FIG. 2, noise was reflected from the open arm of SPST switch (2′) and entered the receiver input. In contrast to the prior art prototype device shown in FIG. 2, in the present technology this noise is absorbed in the first load (6) and does not get to the input of receiver (5). Thanks to this, in the embodiment of the radiometer, there are lower requirement to circulator isolation and it is not necessary to install additionally an isolator as it was made in the prior art prototype (see FIG. 2). This allows almost a double decrease in the sizes of frontend of the radiometer, that is reducing the radiometer dimensions in general, which significantly simplifies radiometer manipulation during an examination, improves convenience of its use, and reduces the time required for an examination in multiple points as is the case, for example, during a breast examination.

Improvement of the measurement accuracy by compensating reflections from the input of antenna (1) in the embodiment of the radiometer is also achieved thanks to receipt in the antenna output of a noise signal from the second load (17). As it has a good thermal contact with the first load (6) through accommodation on one Peltier element, their temperatures are equal Tr₁=Tr₂. But since the noise temperature of the first load Tr₁ is close to the temperature Ta of the noise coming from the antenna output, then the temperature Tr₂ of the second load is close to the temperature of noise Ta at the antenna output. Thanks to this, a fuller compensation of the reflected noise power from the antenna is achieved and the accuracy of measuring the temperature of a biological object is improved.

It should be noted that due to losses in the circulator (4) and SPDT switch (2″), the power of noise coming from the side of the second load (17) onto the antenna outlet will differ from the power of noise coming from the antenna output, therefore, a still fuller compensation of the reflected power is provided by the radiometer embodiment shown in FIG. 4.

In the radiometer embodiment shown in FIG. 4, the noise power from the output of the first microwave load (6) comes on an attenuator (18) connected between the output of the first microwave load (6) and the SPDT switch (2″) and having the temperature radiometer frontend. The noise temperature Tᵣₐ at the attenuator output is equal to

Tᵣₐ=Tᵣ₁*kᵣₐ+(1−kᵣₐ)*T_{amb}, where
kᵣₐ is the transmission coefficient,
T_{amb} is the noise temperature of the radiometer frontend,
Tr₁ is the noise temperature of the first heated resistor (the first microwave load).

The radiometer functions so that the SPDT switch (2″) connects to the first arm of the circulator either the noise signal from the output of antenna (1), the power of which is proportional to the temperature of internal tissues of a biological object, or the noise signal from the output of attenuator (18). The SPDT switch (2″) is controlled by the reference voltage generator (3) having 1 kHz frequency. The noise signal from the output of SPDT switch (2″) passes through the circulator (4) and enters the receiver (5).

The receiver, in the radiometer embodiment shown in FIG. 4, also consists of a low-noise amplifier with bandpass filters (7), an amplitude detector (8), a narrowband low-frequency amplifier (9), and a synchronous detector (10), an integrator (11), a direct current amplifier (12).

At the receiver output, voltage is formed that is proportional to the difference of the noise temperature Ta from the antenna output and the noise temperature Tᵣₐ from the attenuator output:

ΔU=k(Tₐ−Tᵣₐ), where
k is the transmission coefficient of the radiometer
Ta is the noise temperature from the antenna output,
Tra is the noise temperature from the attenuator output.

This voltage is amplified and comes onto the Peltier element (16). Same as in the first embodiment of the present technology shown in FIG. 3, two loads are installed on the Peltier element, which have a good thermal contact with the Peltier element. However, the first load (6) is connected to the input of attenuator (18), which is connected to the SPDT switch, while the second load is connected to the third arm of circulator (4).

The temperature of loads is measured with the help of a temperature sensor (7), which can be installed on the Peltier element and/or at least on one of the loads and has a good thermal contact with them.

Due to negative feedback, voltage at the synchronous detector outlet tends toward zero while noise temperature Tr₁ of the first load comes close to the noise temperature Ta from the antenna output. Due to dissipation losses in the attenuator (18), the temperature of the first and second loads differs from the temperature Ta of noise coming from the antenna output.

$$Tr 1 = Tr 2 = \frac{Ta}{kra} - ( 1 - {k}_{ra} ) \frac{Tamb}{kra} ,$$

where
kᵣₐ is the transmission coefficient of the attenuator,
Tr₁ is the noise temperature of the first load,
Tr₂ is the noise temperature of the second load,
Ta is the noise temperature from the antenna output,
T amb is the noise temperature of the radiometer frontend.

The power of noise coming on the antenna output on the side of the second load (17) is equal to:

Tra=Tr2*kₛk_{cir}+(1−kₛk_{cir})*Tamb, where
kₛ is the transmission coefficient of the switch,
k_{cir-} is the transmission coefficient of the circulator,
Tra is the noise temperature of the attenuator,
Tr₂ is the noise temperature of the second load,
Tamb is the noise temperature of the radiometer frontend.

If the transmission coefficient k of the attenuator coincides with the transmission coefficient of the cascade connection of the circulator and switch kₛk_{cir}, then

Tra=Ta,
that is compensation of the noise reflected from the antenna input occurs.

So, in the design of radiometer according to the present technology, in the modulator a SPDT switch is used instead of a SPST switch, and two microwave loads. Wherein, the first microwave load can be connected to the SPDT switch, the second microwave load is connected to the third arm of the circulator, and the SPDT switch is made with the faculty of connecting to the first arm of the circulator either the antenna or the first microwave load. Besides, between the output of the first microwave load and the SPDT switch, the attenuator (18) is preferably installed, and in such case, the SPDT switch connects to the first arm of the circulator either the antenna (1) or the attenuator.

Such modification of the design of radiometer according to the present technology provides a higher accuracy of the non-invasive measurement of temperature of the inner tissues of biological objects with use for early diagnosis of oncological diseases, also provides reduced dimensions of the instrument, improved convenience of its use, and lower cost of its manufacture.

It should be appreciated that the technology is not limited to the particular embodiments described and illustrated herein but includes all modifications and variations falling within the scope of the invention as defined in the appended claims.

1. A radiometer comprising connected in series
an antenna for contact with a biological subject,
a SPDT switch,
a circulator,
a receiver including:
an amplifier with bandpass filters,
an amplitude detector,
a narrowband low-frequency amplifier
an synchronous detector,
an integrator,
a direct current (dc) power amplifier, and
a reference voltage generator which is connected to the SPDT switch and to the synchronous detector,
a Peltier element, which is connected to an output of the receiver
a first microwave load and a second microwave load mounted on the Peltier element and in thermal contact therewith,
at least one temperature sensor for measuring the temperature of said microwave loads, wherein
the first microwave load is adapted for connection to the SPDT switch,
the SPDT switch is adapted to connect either the antenna or the first microwave load to a first arm of the circulator,
a second arm of the circulator is connected to the receiver, and
a third arm of the circulator is connected to the second microwave load.

2. The radiometer according to claim 1, further comprising an attenuator which is mounted between an output of the first microwave load and the SPDT switch.

3. The radiometer according to claim 1, wherein the temperature sensor is mounted on the Peltier element and/or on the microwave load.

4. The radiometer according to claim 1, wherein the temperature sensor is an infrared sensor for remote temperature measurement.

5. The radiometer according to claim 1 further comprising an integrator which is connected to an output of the temperature sensor.
